(19) United States
(12) Patent Application Publication
(10) Pub. No.: US 2017/0172222 A1
(43) Pub. Date: Jun. 22, 2017

(54) SENSOR MODULE FOR SENSING FORCES TO THE HEAD OF AN INDIVIDUAL AND WIRELESSLY TRANSMITTING SIGNALS CORRESPONDING THERETO FOR ANALYSIS, TRACKING AND/OR REPORTING THE SENSED FORCES

(71) Applicants: Justin J. Morgenthau, South Windsor, CT (US); William G. Eppler, JR., Norwalk, CT (US); William D. Hollingsworth, Wilton, CT (US)

(72) Inventors: Justin J. Morgenthau, South Windsor, CT (US); William G. Eppler, JR., Norwalk, CT (US); William D. Hollingsworth, Wilton, CT (US)

(21) Appl. No.: 15/398,565

(22) Filed: Jan. 4, 2017

Related U.S. Application Data
(63) Continuation-in-part of application No. 15/285,251, filed on Oct. 4, 2016.
Application No. 15/285,251 is a continuation of application No. 14/464,074, filed on Aug. 20, 2014, now Pat. No. 9,462,839.
(60) Provisional application No. 61/881,275, filed on Sep. 23, 2013.
(60) Provisional application No. 61/868,004, filed on Aug. 20, 2013.
(60) Provisional application No. 62/274,575, filed on Jan. 4, 2016.

(51) Int. Cl. A41D 1/00; G01L 5/00; A42B 1/04; A42B 1/24; A61B 5/00; A41D 20/00

(52) U.S. Cl. CPC A41D 1/002; A61B 5/6803; A61B 5/6844; A61B 5/4088; A41D 20/00; A42B 1/041; A42B 1/242; G01L 5/0052; A61B 2562/0219; G08B 29/046

(57) ABSTRACT

Sensor module for sensing forces to the head of an individual and wirelessly transmitting signals corresponding thereto for analysis, tracking and/or reporting the sensed forces.

### CROSS-REFERENCE TO RELATED PATENT APPLICATIONS

This application is a continuation in part of U.S. application Ser. No. 15/285,251 filed Oct. 4, 2016, entitled “SENSOR MODULE FOR SENSING FORCES TO THE HEAD OF AN INDIVIDUAL AND WIRELESSLY TRANSMITTING SIGNALS CORRESPONDING THERETO FOR ANALYSIS, TRACKING AND/OR REPORTING THE SENSED FORCES” which is a continuation of and claims priority under §120 to U.S. patent application Ser. No. 14/464,074, entitled “SENSOR MODULE FOR SENSING FORCES TO THE HEAD OF AN INDIVIDUAL AND WIRELESSLY TRANSMITTING SIGNALS CORRESPONDING THERETO FOR ANALYSIS, TRACKING AND/OR REPORTING THE SENSED FORCES,” filed Aug. 20, 2014. U.S. patent application Ser. No. 14/464,074, entitled “SENSOR MODULE FOR SENSING FORCES TO THE HEAD OF AN INDIVIDUAL AND WIRELESSLY TRANSMITTING SIGNALS CORRESPONDING THERETO FOR ANALYSIS, TRACKING AND/OR REPORTING THE SENSED FORCES,” filed Aug. 20, 2014, is a non-provisional of and claims priority under 35 U.S.C. §119(e) to U.S. Provisional Patent Application Ser. No. 61/881,275, entitled “SENSOR MODULE FOR SENSING FORCES TO THE HEAD OF AN INDIVIDUAL AND WIRELESSLY TRANSMITTING SIGNALS CORRESPONDING THERETO FOR ANALYSIS, TRACKING AND/OR REPORTING THE SENSED FORCES,” filed Sep. 23, 2013; and U.S. Provisional Patent Application Ser. No. 61/868,004, entitled “SENSOR MODULE FOR SENSING FORCES TO THE HEAD OF AN INDIVIDUAL AND WIRELESSLY TRANSMITTING SIGNALS CORRESPONDING THERETO FOR ANALYSIS, TRACKING AND/OR REPORTING THE SENSED FORCES,” filed Aug. 20, 2013, all of which applications are herein incorporated by reference in their entirety. This application also is a non-provisional of and claims priority under 35 U.S.C. §119(e) to U.S. Provisional Patent Application Ser. No. 62/274,575 entitled “SENSOR MODULE AND METHOD FOR SENSING FORCES APPLIED TO THE BODY,” filed Jan. 4, 2016, of which application is incorporated by reference in its entirety.

### BACKGROUND OF THE INVENTION

Field of the Invention

The present invention relates to the sensing of forces to the head of an individual and, more particularly, to the use of a high-quality, mobile physiometric sensor module with a multi-layer distributed data storage, analysis and presentation structure.

Brief Discussion of the Related Art

Individuals engaged in a wide variety of physically demanding sports and activities risk brain or other serious injuries resulting from impact, hyper-extension and other extreme movements or events. Some examples of risk-laden sports include, among many others, football, soccer, baseball, basketball and rugby.

Most attempts to reduce the effects of impacts have included sensors mounted in helmets, in the mouth, or along the side of the head. They do not provide real-time information relating to occurrence of impact events to permit an individual being monitored to be removed from active play for the individual's safety.

### SUMMARY OF THE INVENTION

According to some aspects of the present invention, a sensor is provided that the senses forces applied to the head of an individual where indications of the sensed forces can be transmitted to one or more remote locations permitting visualizations of the force events to which an individual is exposed.

Aspects of the present invention provide accurate sensing of force events and allows data analysis to be performed in real-time and, through more extensive post processing, to permit the warning of players, coaches, parents and others of events which are potentially harmful and could require medical attention. Other aspects of the present invention serve to protect participants involved in sporting events or other activities, including players, coaches, managers and parents, for example, by informing them in real-time of impacts to an individual, assisting them in determining if or when the individual should be removed from the activity for the individual's safety.

Some advantages of different aspects of the present invention include, without limitation, increasing athletic performance while decreasing risk, isolating players who have taken severe or repeated impacts to the head, reinforcement of proper techniques, providing coaches, trainers and parents confidence that they are making a game or activity safer. The sensing device or module, sometimes referred to as a SIM sensor, is carried on or in a support having a shape to surround the head, such as a headband or skull cap, not requiring a helmet or other special equipment, to transmit impacts to the head in real-time. Some applications of the present invention displays data in real-time for athletes on a team as well as for individual use, and stores data historically for each individual being monitored such that the data can be accessed for any time before or after an event for analysis by coaches, trainers, doctors, athletic directors and parents or the like. A software application that can be used to implement aspects of the present invention can allow for functions performed by aspects of the present invention to be activated for the duration of a contact drill in practice such that any subsequent impact that occurs while the system is activated can be saved for later analysis relating to specific drills. Once a particular drill has been completed, head impacts that occurred during the drills can be isolated such that athletes recording the highest G-force impacts can be determined allowing a coach or others involved in the drill to apply special coaching to decrease the amount of impact to a particular athlete's head.

One aspect of the present invention is the positioning of the impact sensor module in alignment with the median nuchal line of the occipital bone of the skull thereby providing extremely accurate data. Positioning of the sensor can be accomplished by placing the sensor module in a pocket formed in a support having a shape to surround the head, such that the sensor module can be comfortably worn during activities at a position to record all impacts and accelerations greater than a preprogrammed set-point. The support can be formed of a headband, a skull cap, or fabric tied around the head like a bandana, and the pocket can be open to facilitate insertion of a sensor module or closed to form the sensor module integrally with the support.

In another aspect, one embodiment of the present invention allows the performance of cognitive and balance evaluation tests to gauge an individual's performance immediately after a possible concussive event in real-time. Balance evaluation tests can be accomplished with the sensor module in place by proper programming of the sensor module or by other equipment coordinating with the sensor module.

Another aspect of the present invention includes a method for monitoring impact forces to the head utilizing a sensor module at the back of the head in alignment with the median nuchal line of the occipital bone utilizing local data service infrastructure and/or global data surface infrastructure.

In a further aspect, the present invention permits monitoring of impact forces to the head of individuals participating in a team activity where a sensor module is worn by each of the participants and a data collection wireless access point receives signals from the sensor modules.

According to one aspect, a sensor is provided comprising a flexible housing comprising a plurality of 3-axis accelerometers, the flexible housing being adapted to be worn by a user and being in contact with a body part of the user, and a module coupled to the accelerometers including a processor which is adapted to sense forces experienced by the body part. In one embodiment, the sensor is constructed within a headband. In another embodiment, the flexible housing comprises at least three 3-axis accelerometers, and wherein the housing comprises flexible couplings between the at least three 3-axis accelerometers.

According to another embodiment, the sensor further comprises flexible circuits that couple the module to two of the at least three 3-axis accelerometers. In yet another embodiment, at least one of the 3-axis accelerometers is positioned at the back of the head of the user in alignment with the median nuchal line.

In another embodiment, the module is adapted to determine rotational acceleration from multiple measurements of linear acceleration. In another embodiment, the module is adapted to receive at least three measurements of linear acceleration from non-colinear points within the sensor.

In another embodiment, the sensor further comprises a sensor adapted to measure rotational velocity. In another embodiment, the sensor is adapted to measure rotational velocity includes a MEMS gyro. In one embodiment, the module is adapted to power on the MEMS gyro responsive to a detection of an event. In another embodiment, the event includes a measurement of an acceleration above a predefined threshold.

In another embodiment, the sensor further comprises a proximity sensor adapted to detect whether the sensor is in contact with the body part of the user. In one embodiment, the module is adapted to power on the sensor responsive to detection by the proximity sensor indicates that sensor is being worn by the user. According to another embodiment, the proximity sensor includes a capacitive sensor pad.

In another embodiment, the housing includes a plurality of rigid circuits associated with each of the plurality of 3-axis accelerometers. In yet another embodiment, the plurality of rigid circuits are coupled by a plurality of flexible circuits. In another embodiment, the sensor further comprises, within the flexible housing, at least one antenna adapted to communicate with one or more external systems. In another embodiment, the sensor comprises, within the flexible housing, a capacitive sensor pad adapted to detect proximity of the sensor to the body part of the user. In yet another embodiment, at least one of the plurality of 3-axis accelerometers is coupled to a rigid circuit associated with the module, the at least one accelerometer being coupled to the rigid circuit by a flexible circuit.

Other aspects and advantages of the present invention will be appreciated from the following description of the invention taken in conjunction with the drawings. The drawings and the following description are meant to be exemplary only of an embodiment of the present invention.

### BRIEF DESCRIPTION OF THE DRAWINGS

FIG. 1 is a perspective showing of a skull relative to a sensor module according to one embodiment of the present invention showing the positioning of the sensor module in substantial alignment with the median nuchal line of the occipital bone of the skull.

FIG. 2 is a plan view of a sensor module according to one embodiment of the present invention with an extended antenna.

FIG. 3 is a perspective view of a headband with the sensor module of FIG. 2 held in a pocket therein.

FIG. 4 is a perspective view of a skull cap on a head and holding the sensor module shown in FIG. 2.

FIG. 5 is a block diagram of a system according to one embodiment of the present invention utilizing a plurality of sensor modules.

FIG. 6 is a diagrammatic representation of the system of one embodiment of the present invention utilized with an athletic field.

FIG. 7 is a plan view of a display of a PDA, such as a smartphone, displaying data obtained with one embodiment of the present invention for an individual.

FIG. 8 is a plan view of a computer display of data obtained with one embodiment of a system consistent with principles of the present invention for a plurality of individuals.

FIG. 9 is a rear view of a headband carrying a sensor module according to one embodiment of the present invention positioned on the rear of the skull of an individual.

FIG. 10 shows a side view of a sensor and headband arrangement according to one embodiment.

FIG. 11 shows a top view of a sensor and headband arrangement according to one embodiment.

FIG. 12 shows an accelerometer arrangement according to one embodiment.

FIG. 13 shows a side view of a sensor and headband arrangement according to one embodiment.

FIG. 14 shows a sensor and headband arrangement according to one embodiment.

### DESCRIPTION OF THE INVENTION

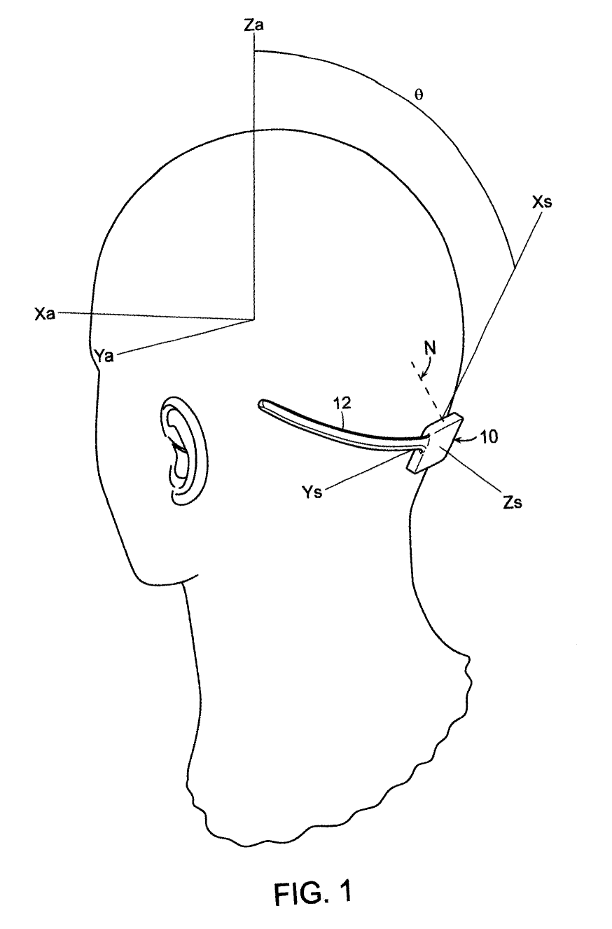

As shown in FIG. 1, a sensor module 10 in accordance with certain aspects of the present invention is typically a small, environmentally sealed device incorporating a sub GHz transceiver, a low power microprocessor, a 3-axis high g accelerometer, a 3-axis low g accelerometer, a 3-axis gyroscope, a non-volatile memory, a battery, a battery charger and other support circuitry. The sensor module 10 is sometimes referred to herein as a mobile sensor or a SIM or an impact monitor. The sensor module 10 is in substantial alignment with the median nuchal line of the occipital bone of the skull shown in dashed lines at N and, normally, between the inferior and superior nuchal lines. One embodiment utilizes a curved elongate antenna 12 extending from the sensor module housing toward the left side of the head. The anatomical axes denoted as Xₐ, Yₐ and Zₐ, the sensor axes denoted as Xₛ, Yₛ and Zₛ and the subtended angle θ are illustrated in FIG. 1. The anatomical axes allow correlation with the axes in the sensor module.

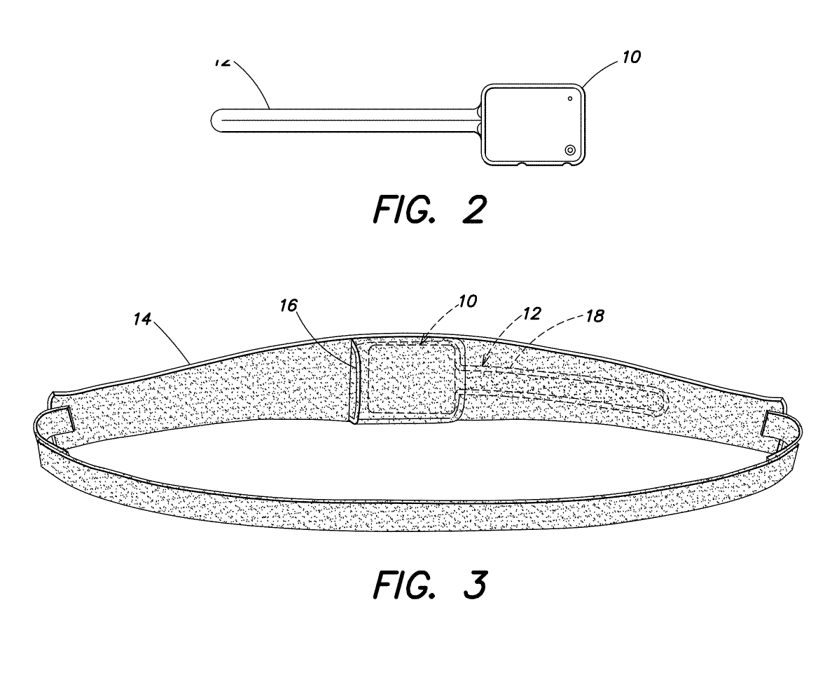

A headband 14 is shown in FIG. 3 and has a pocket 16 arranged along an inner surface or lining and cooperating with an elongated arcuate pocket 18 such that the sensor module 10 and antenna 12 shown in FIGS. 1 and 2 can be inserted within the pocket 16 and arcuate space 18 such that the sensor module is positioned adjacent the skull. The headband is preferably made of a non-stretchable material having only a small section thereof made of elastic to allow for form fitting. The headband thus stabilizes the sensor module and prevents “double hit” sensing by keeping the sensor module firmly in place against the skull. The outer surface of the headband adjacent the pocket 16 can carry indicia I to facilitate accurate location of the sensor module on the head. The indicia can also include an arrow to make certain that the headband is properly oriented.

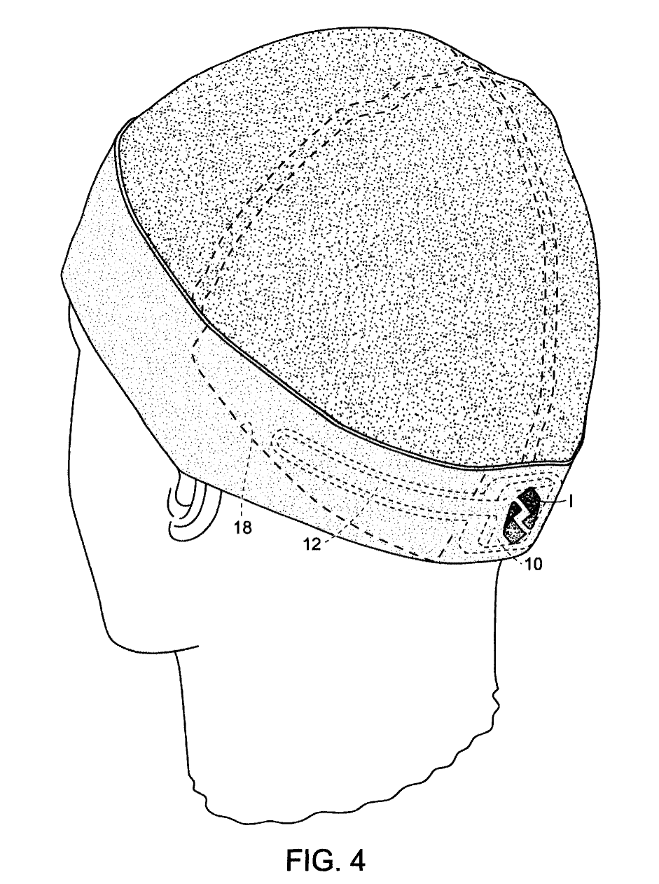

A skull cap having a structure around the periphery including the pocket structure described above is shown in FIG. 4.

In one embodiment, the sensor module communicates with an access point in a wireless fashion such as over the 915 MHz ISM band in the U.S. Other bands are possible through minor firmware and hardware changes over the frequency range of 300 MHz-348 MHz, 389 MHz-464 MHz and 779 MHz-928 MHz. The sensor module 10 is capable of measuring linear acceleration events up to +/−400 G and rotational velocities up to +/−2000°/sec at a 1 KHz sample rate. An “event” is defined as a 3-axis G recording of 10 ms before and 52 ms after a threshold is exceeded. The threshold is calculated as (xg²⁺y_{g}²⁺zg²) and is adjustable. When an event is detected, the event is transmitted wirelessly in real-time (within a few tenths of a second) to the access point.

If wireless communication with the access point is interrupted, the event is stored in internal non-volatile memory. When wireless communication is restored, any saved events are transmitted.

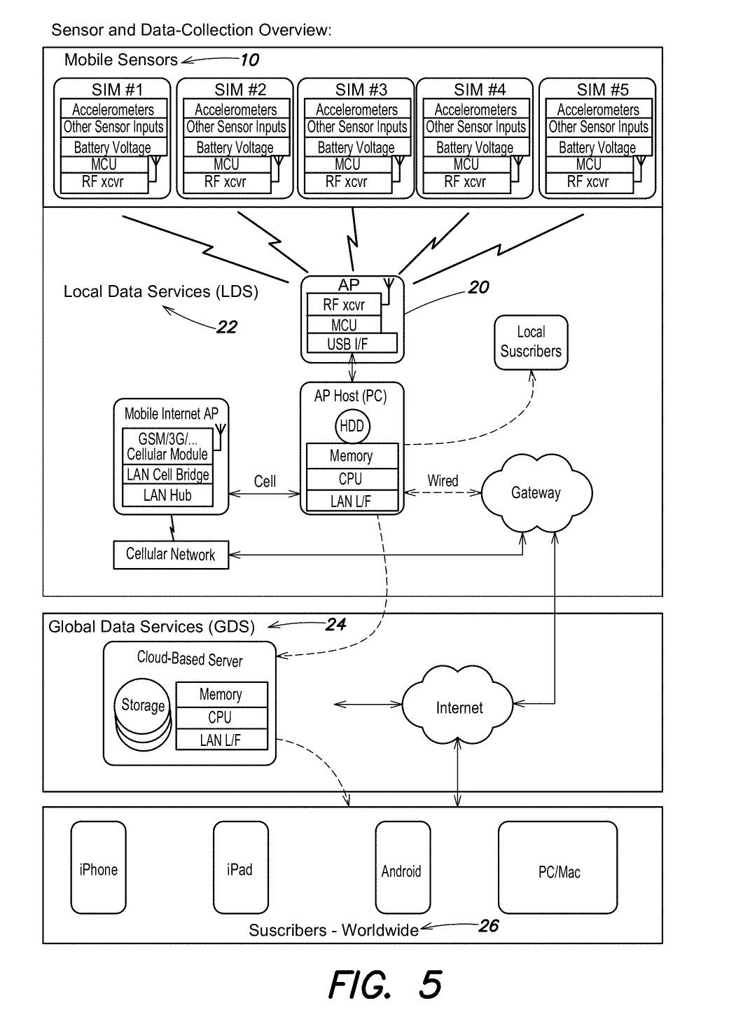

As shown in FIG. 5, the system of one embodiment of the present invention includes, in an exemplary embodiment, a plurality of sensor modules each in communication with an appropriate access point 20. Multiple impact monitors 10 can be used concurrently with a single access point 20. The access point and its associated impact monitors are assigned primary and secondary communication channels (from a set of over 30 for the 915 MHz band). If communication is not established on the primary channel within a few seconds, the impact monitors try on the secondary channel. This procedure is repeated until communication is established. The communication protocol is packet based with robust error checking/correction to increase the likelihood of valid data exchange. Each packet includes globally unique source and destination device identifiers to further insure data integrity. Each ‘event’ packet is tagged with a time stamp for unambiguous correlation of the data ‘event’ with the physical event producing it.

The local data services infrastructure 22 and the global data services infrastructure 24 all achieve the data integrity goal by holding all measurements until they have been successfully and verifiably transmitted to the next stage in the system.

The system according to one embodiment of the present invention is formed of three main subsystems as shown in FIG. 5.
1. Mobile sensors 10 (SIMs, sensor modules).
2. Local Data Services 22 (LDS) infrastructure:
   Data collection wireless access point (AP).
   Local data storage.
   Local data services (analysis, formatting and presentation).
   Local administrator and account services.
3. Global Data Services 24 (GDS) infrastructure:
   Cloud-based server facilities, essential for reliability and scalability.
   Data storage and perpetual archival and back-up.
   Data analysis, formatting and presentation.
   User-account services and revenue management.

In addition, subscribers 26 (local and global) represent the final consumers of all available analytics.

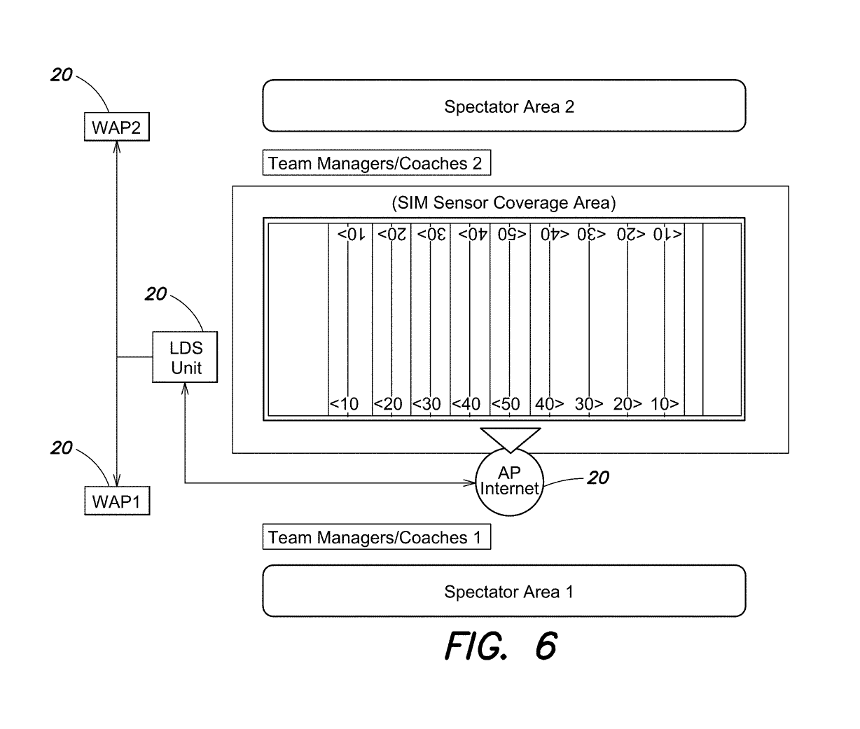

The diagram in FIG. 6 shows a typical football field, with the system installed at the sidelines. In this example, there can be one (shown) or more WAP (WiFi Access Points) 20 to provide adequate WiFi coverage to both sides of the playing field (staff and spectators). Staff for both teams have their own display devices (iPAD, etc.), and are granted access to their respective team's information only.

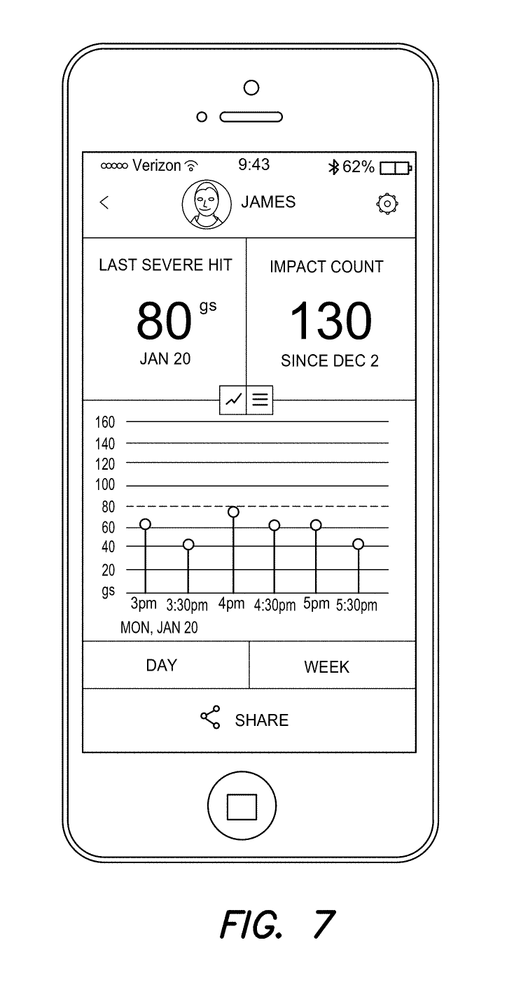

The sensor modules each collect data on impact events to the wearer's head that occur during typical sports activities (football, soccer, etc.). The sensor data being recorded includes 3-axis linear accelerometer data, 3-axis rotational data, diagnostics and status, time stamp, and individual device identification as shown in FIG. 7. The sensor modules (SIMs) also contain a small processor that handles sensor data acquisition and manages a wireless radio link with the AP. The SIMs can incorporate a wider and more extensive range of sensor inputs, including standard health monitoring functions (heart rate, respiration, temperature, GSR, etc.) and other physiological parameters.

Impact-event data from the sensor modules are transmitted to the nearby access point via a low-power 900 MHz radio link. The data is received by the AP, processed and presented almost instantaneously to nearby coaches/administrators through the LDS. The LDS infrastructure includes the AP, plus a local computer (PC). This subsystem primarily serves as a real-time data collection and storage unit.

The LDS can be physically deployed at the sidelines, as a mobile LDS or as a fixed LDS at a given sports complex or playing field/stadium/court. In either case, the functionality of the LDS remains the same:
The AP function block provides the RF link to communicate with all SIM devices within the sports arena.
The AP streams all SIM data to the LDS unit controller (PC).
The LDS controller provides bulk local storage for SIM data.
The LDS controller also provides a limited range of analytics, formatting and presentation services.
   Without an internet connection (access to the GDS), analytics would be limited to the data currently stored in the LDS.
   Local user-access would be via a local WAP device (WiFi Access Point). The analytics are accessed and presented using a common web- GUI interface, using a typical web-browser on a laptop or tablet (or iPhone, iPad, etc.).
   Optionally, the user access can be a custom iPhone/Pad application, rather than using a browser interface. A custom iOS/Android application can be used.
   The LDS services are generally meant for use by the nearby coaches and administrative staff.

The LDS should be connected to the global internet (and thus, the GDS) whenever possible. However, the reality is that many sports venues (football fields, soccer fields, etc.) have little or no access to the global internet, and often lack even AC power.

As an option to a direct internet connection, the LDS can utilize commonly available “LAN/CELL” bridge devices, which allow the use of public cellular networks (GSM, 3G, 4G-LTE, etc.) as the gateway to the internet (and therefore, the GDS). The LAN/CELL bridge devices are generally compatible with a wide range of cellular networks. In most cases, all that is required is a prepaid cellular card plugged into the LAN/CELL bridge unit.

The physical implementation of the LDS has as basic elements, options for fixed or mobile deployments, AC or solar power, battery power, LAN hub, WAP (WiFi-AP), and a cellular-LAN bridge device.

Some of the features of the present invention include

For the Mobile LDS:
Rugged, weather-proof enclosure, suitable for portable hand-carried usage
Carrying handles.
Locking cover(s).
PC based, with integral high-reliability storage units (preferably SSD), able to withstand the rigors of mobile use at sporting events.
Internal battery supply, sized to provide at least 8 hours of run-time.
Battery AC charging port: Accepts AC line-voltage input.
Battery DEPENDENT CLAIM charging port: Accepts typical automotive 12 VDC (nominal) input.
Video output port: VGA/HDMI/DVI, for attaching a direct console display.
Antennae port.

For the fixed LDS:
Rugged, weather-tight enclosure, suitable for outside use.
Mounting flanges and fixing hardware suitable for mounting to walls, poles, ceilings.
Locking cover(s) with security or tamper-evident features and enclosure-access alarm switch.
PC based, with integral high-reliability storage units (possibly SSD).
Able to withstand considerable temperature extremes.
Internal battery supply, sized to provide at least 2 hours of run-time.
AC input port, for normal operating power.
An on-board charger to keep the internal battery charged in case it's
needed.
Video output port: VGA/HDMI/DVI, for attaching a direct console display.
Antenna port.

For networking options:
LAN port so the LDS can connect directly to a 10/100/1G LAN network.
WiFi-node so the LDS can connect to a camput-wide wireless network as a client.
WAP (WiFi AP) so the LDS can provide a local WiFi “network cloud” and the LDS-generated analytics can be accessed locally by coaches on their own laptops or other devices.

The Global Data Services (GDS) subsystem can be considered “cloud based” insofar as it exists as a collection of stored sensor data, programs, and the physical computing hardware could be provided by any number of service providers in this field.

There are many advantages of implementing a “cloud based” design rather than using fixed in-house server hardware implemented using commodity PCs. The key elements of a cloud based strategy can be summarized as follows:
Location: Server hardware and related data storage facilities can be placed nearly anywhere in the world, wherever operating costs and network accessibility are optimal for the application.
Reliability: Cloud servers offer much higher operational reliability, and often feature auto-failover to on-site (or remote) backup servers. Failover events are usually transparent to the hosted applications and any attached users.
Data backups: Automatic backups of data and programs. Proper procedures and facilities management ensures data integrity and security, for both on-and off-site backup archives.
Scalability: As the underlying dataset grows, and the number of attached users increases, the server architecture will need to scale u accordingly and do so in a manner that does not require a major redesign of either the dataset or the related application programs.
   At the low end, just a fraction of one server (PC) may be utilized by employing a virtual OS “slice” of the available computing power of that one PC.
   As requirements grow, dedicated servers and even multiple servers can be utilized to share the attached-user load and access to huge perpetual datasets.
Network access: A large cloud based server will have dedicated top-tier access to the global internet. This will be necessary to efficiently handle the expected number of subscribers.
Infrastructure: The facilities, power, cooling and security are all managed and cost-optimized not just for one or a few servers, but for an entire server-farm encompassing potentially many thousands of servers.
Site Backup: High availability cloud service providers often provide geographically diverse locations. This enables a rapid cutover and recovery from catastrophic events (earthquakes, floods, etc.).

The SIM sensors, AP+LDS, and GDS, together form a system whose primary purpose is data collection, storage, analysis and presentation.

A key element of the system is the acquisition and perpetual long-term storage of all available sensor (SIM) data. Over time, no doubt there will be many ways of analyzing that data for various purposes. Sometimes for the user's own personal “performance monitoring” needs. At other times, the data will be invaluable for analysis of athletic performance and related injuries, correlating with demographics other recorded factors.

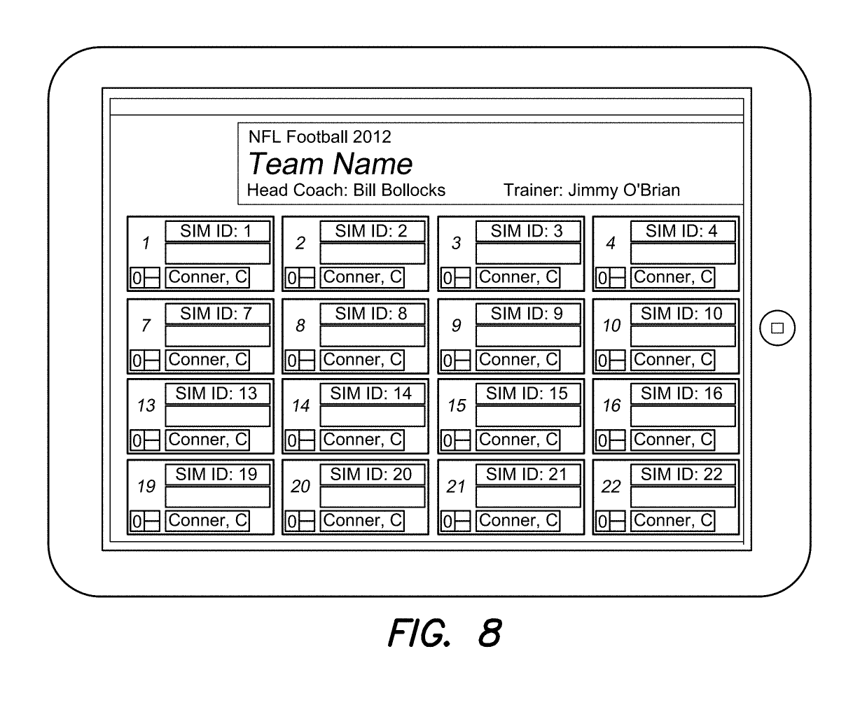

FIG. 8 shows a user interface which can be used as an exemplary layout of a sensor-event record, as it would be stored (locally) in the LDS, and transferred to/from the GDS (and stored there as well). The sensor-event record, as shown, contains discrete fields which are, in most cases, simply extracted from the raw sensor-event data (as delivered over the RF link). These discrete fields are brought out so that the LDS/GDS database engine (mySQL, etc.) can use those fields to efficiently index and organize the records. Whether the data storage (on disk) is a “relatively small” database like on the LDS, or scaled up to “multi-terabyte” database (on the GDS), it is important to bring out some fields like this because the database engine is most efficient at what it does best-indexing and accessing data organized into fields. On the LDS there will be a single SQL (or other) program managing event records. On the GDS, the equivalent “SQL engine” function can easily be scaled up to many servers, all accessing the same storage unit, providing analytics for many thousands of users worldwide. Keeping the event-record the same everywhere keeps things uniform. The system relies not only on the sensor data, but a number of interrelated databases which ensure the proper identification, storage, categorization, analysis and distribution of the results. The sensor event records are stored and managed by the database engine (SQL, etc.), using one or more of the “-ID” fields as primary index keys. The user database contains detailed user identification (name, address), and a list of all SIM-ID/IDX's that have been assigned to this user.

Each organization (school, university, club, etc.) will be registered into the system, and each organization will be responsible for one or more AP+LDS units. Each AP+LDS unit will be registered and activated before it can participate in the system. This is mainly to prevent the use of unauthorized copies of the LDS.

The subscriber database authenticates the final consumers of the sensor data and its derivative analytics. Subscribers are pay-for-access users, and therefore a related mechanism will be the billing and user-account management for each subscriber. There will be various subscriber access levels.

The most common access method, generally compatible with most if not all devices, is a typical browser-based GUI. It would be accessed by a fixed URL. The browser interface GUI should be straightforward and as simple as possible in terms of using the “special features” of any particular browser. In fact, all analytics should be delivered as graphic images (JPEG/GIF/PNG) that are computed and delivered as needed. Some of the browsers to support include:
IE (Microsoft, version 6+)
Safari (Apple)
Opera (PC and mobile)
Google Chrome
Firefox

The browser GUI interface couldimilar to the “large tablet” version of the iOS/Android apps, taking full advantage of a much larger screen. Also, browser access usually means that printing of analytics will be possible.

The following is a general description of data flow activities within the LDS:
1. The LDS Windows-app:
   a. Receive sensor data from the AP (RF-link).
   b. Unscramble or otherwise decrypt, then validate, the data.
   c. Create standardized “sensor event records”.
   d. Store these records on the local hard-drive using the resident database engine (mySQL, etc,).
   e. Act as an admin-console for configurations settings in the system.
   f. Generates requested analytics from the local database.
   g. Cache all requested analytics. These will be used locally by the web-server and app-server delivery subsystems.
   h. Upload any new sensor-event records to the GDS.
      i. Local sensor-event caching should have an admin-configurable “cache size” setting. Usually it will be set to “limited to disk space”, but in some cases it might be “limited to the last 12 months of data”.
   i. Download sensor-event records from the GDS, for any analytics-requests which require sensor-event records which aren't already stored locally.
   j. Manage user-registration (assignment of SIMs).
   k. Manage user and subscriber authentication.
      i. Download account data and credentials from the GDS whenever possible.
      ii. It will be necessary to locally cache user/subscriber credentials, since the LDS will likely not have a permanent internet connection to the GDS.

One or more of the following software capabilities can be used:
2. A resident web-server will serve analytics to locally connected (via LAN or localized WiFi cloud) subscribers that are accessing the system using a web- browser.
3. A resident iOS app-server will serve analytics to locally connected (via LAN or localized WiFi cloud) subscribers that are accessing the system using an iOS device.
4. A resident Android app-server will serve analytics to locally connected (via LAN or localized WiFi cloud) subscribers that are accessing the system using an Android device.
5. A resident Windows Phone app-server will serve analytics to locally connected (via LAN or localized WiFi cloud) subscribers that are accessing the system using a Windows Phone device.

The following is a general description of programs running on the GDS (via a Cloud Service):
1. Operating system.
2. A database engine.
3. LDS host-side server module.
4. Web server module.
   a. Any web server-related plug-ins and support programs (PHP, Perl, Java, Python, etc.) that may be necessary.
   b. The custom “website” (HTML and support files), designed to implement a web-based GUI. This would be designed to look very similar (but not identical) to the LDS version.
5. iOS Application Server module.
6. Android Application Server module.
7. Windows Phone application server module.

The following is a general description of activities within the GDS:
1. LDS host-side server.
   a. Manage connections to remote LDS units.
   b. Upload/download sensor even records, as requested by the remote LDSs.
   c. Store/retrieve these records using the resident database engine (mySQL, etc.).
   d. Generates requested analytics from the local database.
   e. Cache all requested analytics. These will be used by the web-server and app-server delivery subsystems.
   f. Manage user and subscriber authentication as requested by the remote LDSs.
   g. Interface with the subscriber billing and account management system.
2. The resident web-server will serve analytics to internet-connected subscribers that are accessing the system using a web-browser.
3. The resident iOS app-server will serve analytics to internet-connected subscribers that are accessing the system using an iOS device.
4. The resident Android app-server will serve analytics to internet-connected subscribers that are accessing the system using an Android device.
5. The resident Windows Phone app-server will serve analytics to internet-connected subscribers that are accessing the system using a Windows Phone device.

There are many possible ways of analyzing sensor-data, from real-time events (at a football game), to more generalized statistical research.

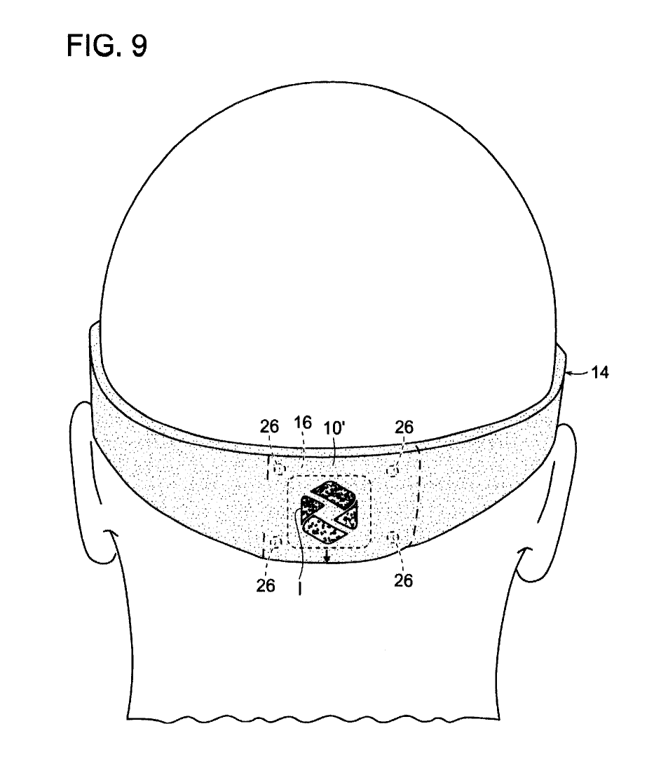

A variation of one embodiment of the present invention is illustrated in FIG. 9 wherein the sensor module 10′ has an antenna within the housing thereof such that an arcuate space for the antenna in the headband is not required. Additionally, arrow indicia is displayed on the outer surface of the headband at the pocket receiving the sensor module 10′ to assure that the individual wearing the headband has vertically properly aligned the headband and the accompanying sensor module. Additionally, portions of non-Newtonian fluid are positioned on the inner surface of the headband to separate the skull from the sensor module. The non-Newtonian fluid, in one example, will be supplied in four small ovals sewn into the inner lining of the headband SIM pocket. The non-Newtonian fluid serves as a small buffer against the SIM and the back of the head which will allow the SIM to generate a more accurate impact reading.

From the above, it should be appreciated that certain aspects of the present invention permits continuous sampling and recording of high-g accelerometer and gyro data since, when an impact/event is detected, the data that was recorded at the impact point is transmitted along with data relating to what happened before the impact. More particularly, high-g accelerometer (linear) and gyroscope (rotation) are sampled/monitored at, for example, a 1 KHz rate and successive samples of the linear and rotational sensor data are placed in a circular buffer. One system consistent with principles of the present invention can be used in conjunction with specialized software to perform a cognitive and balance evaluation test when data indicates that such tests are desirable.

The above described embodiments of the present invention can be varied as will be understood by one of ordinary skill in the art, for example, use of different radio frequencies and radio transmission chips and circuits for data transmission, inclusion of additional sensors and sensing capabilities within the sensor module, use of alternative power sources permitting charging mechanisms such as induction charging, and motion-based energy “harvesting”. Additionally, the present invention can utilize cell phones, tablet computers, laptop computers or other similar devices as an alternative to a dedicated LDS system for example using Bluetooth or WiFi for communication with the sensor modules, the use of a self-contained LDS system including integral computing capability but not including an external laptop computer device, a system using a “self-contained” LDS incorporating some elements of functionality from the GDS to allow use without a GDS system. Alternative designs could also utilize a general purpose network technology (rather than one specifically deployed for the application of the present invention within an LDS) examples of which would be a WiFi network, cellular phone or paging network and a general purpose data communications network such that alternative designs could include a system without and LDS but where some of the functionality of the LDS is moved to the GDS to allow correlation with the axes in the sensor module.

There are many issues with determining whether an event has occurred in sensing whether an individual has incurred a concussive force. Various additional embodiments described herein address some of these issues within a sensor and associated systems for analyzing, tracking and/or reporting sensed forces, and for providing better sensors, systems and methods for determining such forces. For instance, one issue with existing sensors is that wearable acceleration sensors worn external to the body are susceptible to false events from direct contact with the sensor. Filters based on frequency analysis, heuristics, and machine learning are only a partial solution, and additional improvements are needed. According to one aspect of the present invention, multiple 3-axis sensors are provided that are spaced around the head with a flexible headband. It is appreciated that with such a system, a high degree of correlation between the sensors indicates it was the head that moved, and not just the sensor.

It is also appreciated that there are problems with wearable acceleration sensors in that they are susceptible to significant noise when measuring rotation, rendering rotation measurements highly inaccurate. A primary cause for this is “waves” in soft tissues of the person's body located under the sensor causes oscillating rotation. Accurate rotation measurements are not only required to determine rotation forces on the brain, but also to translate the linear acceleration measured at the sensor to a measurement of forces at the center of the head.

According to one aspect, it is appreciated that such oscillations may be eliminated by spacing three (3) or more accelerometers around the head and looking at differential linear accelerations to determine rotational acceleration. This effectively spreads the measurement out across the span of the sensors, eliminating the noise susceptibility of measuring at a single point. In particular, waves have a significantly reduced effect when measuring across multiple points spanning the head as opposed to measuring at a single point. In a single-point measurement, the waves create “wobble” in the sensor, which is only limited by the relatively small footprint of the sensor. By distributing sensors around the head, a much larger “virtual footprint” may be created for the sensor, which averages out the small localized effects of waves in the skin.

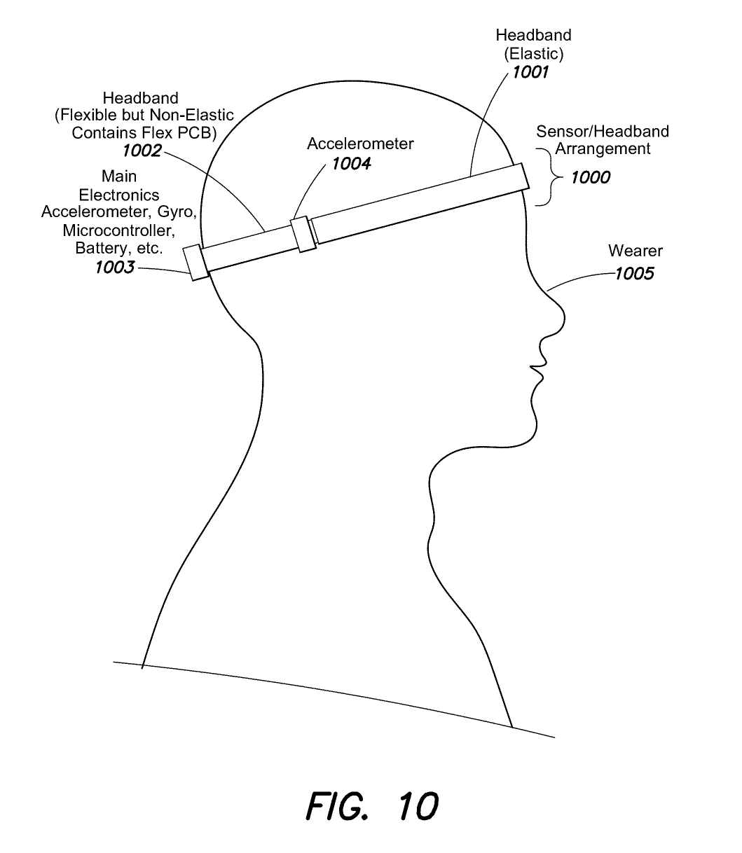

FIG. 10 shows an example configuration of a sensor (side view) and headband arrangement 1000 according to one embodiment. As shown in FIG. 10, multiple three (3)-axis accelerometers (e.g., accelerometer 1004) are used and located within the headband to more accurately detect forces applied to the head. Optionally, the sensors are not all located within the same plane such that component forces in different directions can be more accurately sensed. As shown in FIG. 10, the main electronics 1003 may be located in a portion of the headband located at the back of the head, and additional 3-axis accelerometers are located on each side of the headband, located behind the ears of the wearer (e.g., wearer 1005). The headband may include a portion that is flexible but non-elastic (e.g., portion 1002), and the portion may include a flexible printed circuit board (PCB). The headband may also include an elastic portion (e.g., portion 1001) that holds the flexible portion to the head of the wearer.

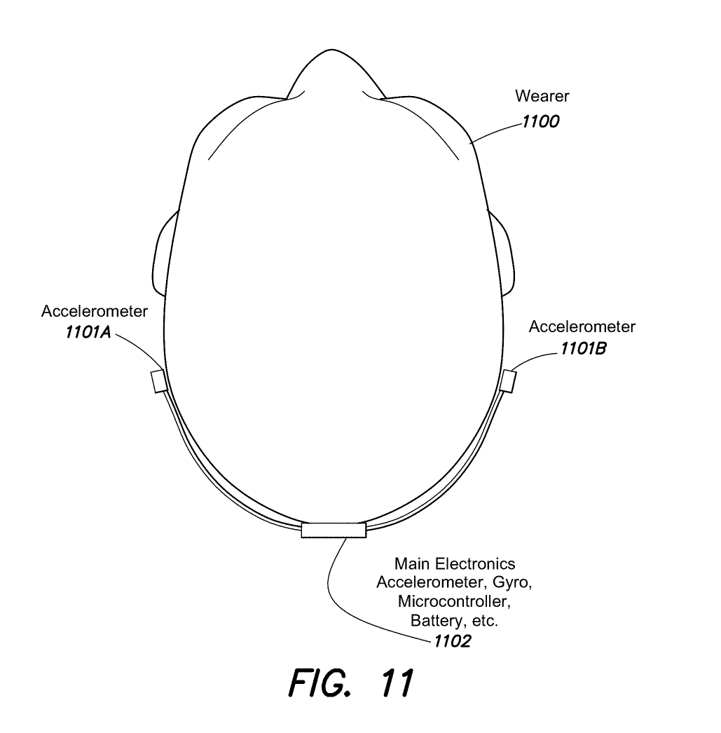

FIG. 11 shows a top view of the location of sensors within the headband. As shown, the headband may be worn by a wearer 1100, and the headband may include sensors located near the back of the ears (e.g., accelerometers 1101A, 1101B). Also, the main electronics (e.g., element 1102) may include an additional accelerometer, a gyro, microcontroller, battery, among other elements.

As discussed above, there may be additional computations that may be required when using multiple 3-axis sensors. More particularly, it may be desired to determine the rotational acceleration on the head, as it is appreciated that damaging forces to the head are correlated with rotational acceleration. The following is an example calculation:

### Rotational Acceleration Computation

The following points are defined in 3-dimensional space:

L—location of the left accelerometer

R—location of the right accelerometer

B—location of the back accelerometer

C—the midpoint between L and R

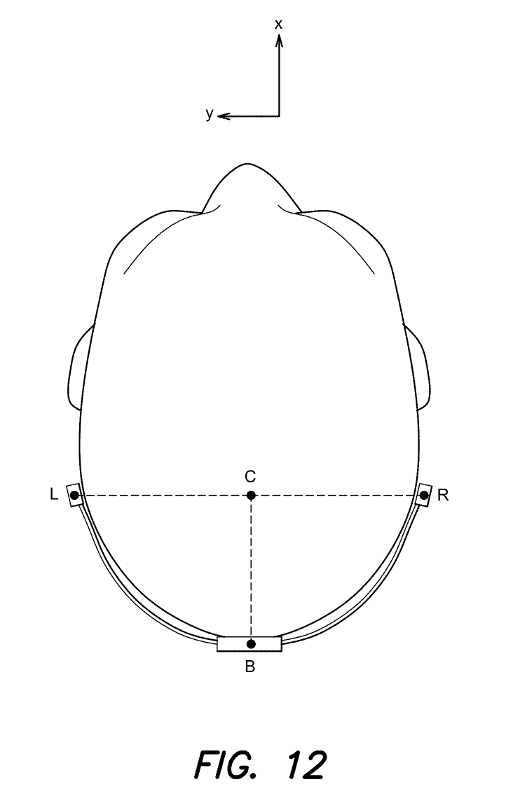

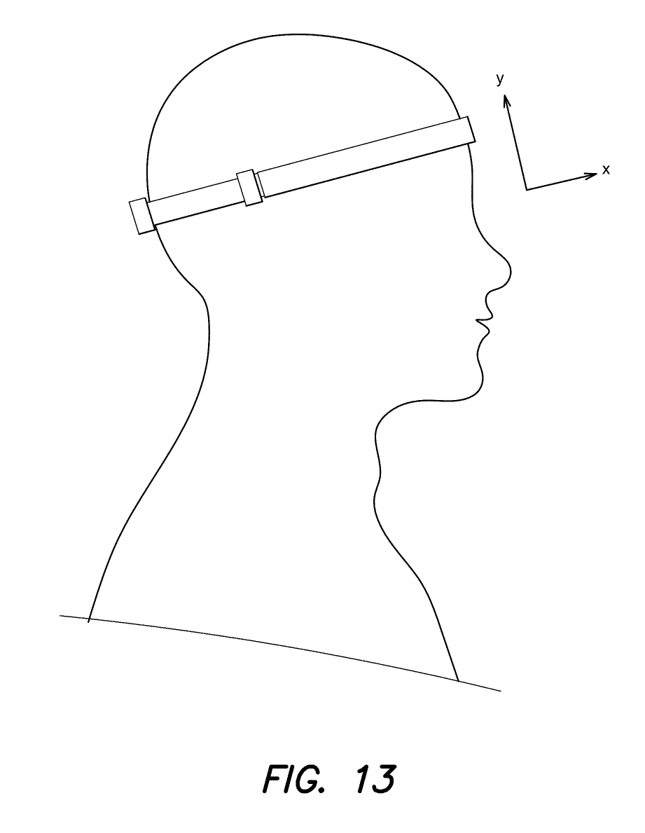

Also defined is a coordinate system where all accelerometers lie in the x-y plane, the x-axis runs through B, the y axis runs through L and R, and the z axis points up (e.g., as shown in FIGS. 12 and 13).

We can then compute the rotational acceleration about each of the three (3) axes using the following equations:

$${a}_{C} = \frac{{a}_{L} + {a}_{R}}{2}$$

$${α}_{x} = \frac{{a}_{zR} - {a}_{zL}}{\vert \vec{RL} \vert} - {ω}_{y} {ω}_{z}$$

$${α}_{y} = \frac{{a}_{zB} - {a}_{zC}}{\vert \vec{BC} \vert} - {ω}_{x} {ω}_{z}$$

$${α}_{z} = \frac{{a}_{xR} - {a}_{xL}}{2 \vert \vec{RL} \vert} - \frac{{a}_{yB} - {a}_{yC}}{2 \vert \vec{BC} \vert}$$

Where:
aL, aR, and aC are the linear accelerations at L, R, and C respectively
α is the rotational acceleration
ω is the rotational velocity

Multiple Accelerometer Data Processing Flow

Below is described an example process for processing data from multiple accelerometers:
1) An event is triggered by all three accelerometers reading above a threshold (typically, for example, 16G for athletics).
2) The event data comprises accelerometer samples for a period of time pre-trigger and post-trigger. For athletics, this is approximately 10 milliseconds of pre-trigger data and 50 milliseconds of post-trigger data.
3) The 3-axis data from the Left and Right accelerometers is transformed into the coordinate system of the Back accelerometer. The typical arrangement will have the Left and Right accelerometers rotated about the Z-axis relative to the Back accelerometer. In this case, the transformed acceleration samples will be computed by first creating a rotation matrix for each sensor as follows:

$${R}_{z} ( θ ) = [ \begin{matrix}cos θ & - sin θ & 0 \\ sin θ & cos θ & 0 \\ 0 & 0 & 1\end{matrix} ]$$

Where θ is the angle of rotation about the Z-Axis.
Each acceleration data point is then multiplied by this matrix to compute the transformed data in the common coordinate system.
4) The acceleration data from each accelerometer can be compared using a correlation function (such as the Pearson product-moment correlation coefficient). If the three measurements have high correlation (above some specified threshold) then the hit can be considered valid. According to one implementation, if 2 out of 3 of the measurements are highly correlated, then the event is processed with those 2 measurements, with the non-correlated measurement discarded. This corresponds to the case where one of the sensors was directly contacted during a real head impact. It should be noted that processing an event with only 2 sensor measurements may result in degraded accuracy, particularly in the rotational acceleration calculation.

It should be appreciated, however, that any number of accelerometers may be used to increase the accuracy of the sensor.

There also exists a problem of how to build a headband with multiple accelerometers. For instance, it is appreciated that it would be desirable to have a headband that includes multiple accelerometers that have non-rigid mechanical coupling to each other through the headband such that they achieve mechanical coupling via the wearer's head. In this way, the multiple accelerometers have a higher chance of detecting an applied head force rather than a force applied to one of the other accelerometers or from the headband.

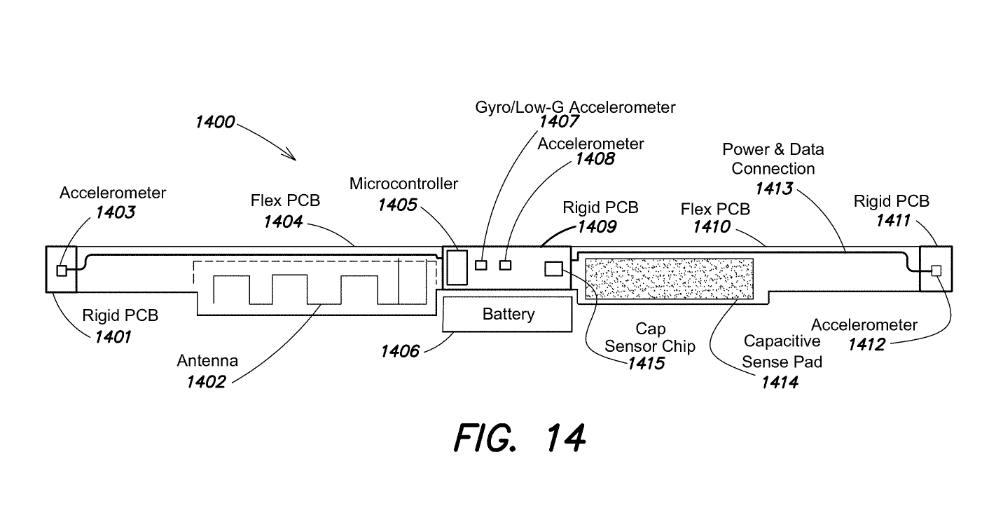

One embodiment of the present invention relates to a rigid-flex circuit with rigid portions to hold accelerometers and other circuit components at the back of the head and approximately above each ear, with flexible portions connecting them. The circuit may be bonded to a fabric backing and/or encapsulated in a flexible plastic housing for strength, durability, and protection from sweat and the elements. FIG. 14 shows an example layout of such a rigid-flex circuit within a headband.

In particular, FIG. 14 shows an example headband/sensor arrangement 1400 including a number of rigid PCB elements connected by flexible PCB sections. In particular, a rigid PCB section 1401 is coupled to a rigid PCB 1409 that houses main processing components through a flexible PCB section 1404. The rigid PCB section 1409 may house the battery, a microcontroller 1405, a gyro/low-G accelerometer 1407, a three-axis accelerometer (e.g., accelerometer 1408), capacitive sensor (e.g., capacitive sensor chip 1415), and battery 1406, among other components. In one embodiment, antennas (e.g., antenna 1402) is embedded within a flexible PCB portion. In another embodiment, a capacitive sensor pad (e.g., capacitive sensing pad 1414) is positioned within the flexible PCB portion (e.g., flex PCB 1410). Flexible PCB portions may also include one or more connections to one or more components (e.g., via power and data connection 1413). Another rigid PCB section (section 1411) may include a third three-axis accelerometer 1412.

According to another embodiment, accelerometers 1403, 1412 are positioned accurately within a defined plane because they are positioned within the flexible/rigid PCB component which is held to the wearer's head (e.g., via an optional headband). Rigid flex printed circuit boards are boards that may use a combination of flexible and rigid board technologies in an application. Most rigid flex boards comprise multiple layers of flexible circuit substrates attached to one or more rigid boards externally and/or internally, depending upon the design of the application.

The flex portion of the assembly may be manufactured, for example, on a flexible base material, such as polyamide film. Metal layers are attached to the base layer to create the conductive layers, either by applying metal foil with an adhesive or electroplating or other method. Multi-layer flex circuits may be created, for example, by laminating multiple layers together. However, it should be appreciated that other flexible materials, shapes of the sensor and elements, and solutions may be used (e.g., flexible conductive fabric), and that certain aspects of the invention are not limited thereto.

Described below is an example process of making a measurement, and how is noise eliminated or reduced.
First, the linear acceleration at each of the 3 sensors is measured for the duration of the event.
Next, the rotational velocity and acceleration of the head is computed at each discrete time step during the event. (see below).
Finally, the acceleration at the head's center of mass is computed using the linear acceleration measurements and the computed rotational acceleration and rotational velocity using the following equation:

a_{C}=a_{S}+ω×(ω×\vec{CS})+α×\vec{CS}
Where
a_{C} is the accleration at the Center-of-Mass
a_{S} is the acceleration at the sensor
ω is the rotational velocity
α is the rotational acceleration
CS is the vector from the sensor to the center-of-mass

Equations to determine rotational acceleration from linear acceleration at multiple points (e.g., the equations for computing rotational acceleration about each of the three (3) axes as discussed above) requires one of the following:
a) measurements at a minimum of 4 non-coplanar points
b) knowledge of the rotational velocity during measurement

However, it is appreciated that a 4 non-coplanar point sensor arrangement solution is generally not practical (e.g., a sensor located on top of the head or under the chin may be required), and thus knowledge of rotational velocity may be needed. One solution to this is that all points in a headband are roughly coplanar (e.g., as provided by the rigid/flex PCB arrangement), so to have knowledge of the rotational velocity, one implementation may use a MEMS Gyro sensor. This sensor may encounter noise during an impact event, however it can be used to determine the starting and/or ending conditions. The rotational velocity during the event can be determined by integrating the rotational acceleration derived from linear acceleration measurements, and applying this to either the starting or ending conditions measured by the gyro, as discussed above. Events may be triggered, for example, from linear accelerations exceeding a defined threshold.

Also, as discussed, the starting conditions may be determined using the gyro, and then the linear accelerometer data may be used to determine rotational velocity. In particular, given a rotational velocity starting condition, the equations above can compute the rotational acceleration. This rotational acceleration can then be used in a discrete time integration to determine the rotational velocity at the next sample time. This process is then repeated to compute rotational velocities and accelerations for the entire event duration. This process is symmetric, so the same computations can be done working backward from a measurement at the end of the event as described in detail below.

One issue relating to the use of a MEMS gyro includes the issue that MEMS gyros are power-hungry components (relative to other components in the headband) and therefore the use of the MEMS gyro reduces battery life significantly. One solution to this issue includes keeping the gyro powered down until a high-G event is detected. At this point, the system powers on the gyro and starts taking measurements. Most commercially-available MEMS gyros take 30-100 milliseconds to start producing valid data after power on. It is appreciated that when a valid rotational velocity measurement from the gyro is achieved, it is possible to work backward from this point iteratively using differential measurements from the linear accelerometers to determine the history of rotational velocity and rotational acceleration during the impact event.

The high-G event may be initially detected as described above, using the accelerometers. For instance, the high-G event may be defined as any event where all three accelerometers measure an acceleration above some specified threshold. The severity of the event may be detected using metrics such as Peak Linear Acceleration (PLA) and Peak Rotational Acceleration (PRA). Other metrics could be envisioned that take into consideration the duration or total energy of the event.

Below is a description of the example calculations that can be used to trace back the event:
This is a discrete-time integration in reverse, which can be approximated using the following function:

ωₜ₋₁=ωₜ−αₜΔt
where:
ωₜ₋₁ is the rotational velocity at step t−1
ωₜ is the rotational velocity at step t
αₜ is the rotational acceleration at step t
Δt is the time interval of each step

Another issue with sensor operation is that it would be beneficial to know whether a headband (or any other type of body sensor device) is being worn at a given time, both to turn the device off when not in use, as well as to filter events that may occur during transport and storage. It is appreciated that an additional sensor may be used to determine whether the device is being worn by a user. For example, in one implementation, a low-power capacitive proximity sensor can be used which can detect changes in electric field when an object is placed next to the sensor area. Furthermore, a sensor which can measure electrical permittivity can discriminate between placement next to the body and placement next to other objects, such as a table.

The sensor may be located, for example, as shown above within the headband. It may include a capacitive sense pad embedded within one of the PCB elements of the headband. In particular, the sensor pad etched may be into the conductive layers of the PCB. This pad may be connected to a capacitive sensing chip, which controls and monitors the state of the sensor pad. This chip interfaces with the microcontroller via a communications bus (I2C or SPI) and/or via general-purpose I/O signals.

Be implementing a proximity sensor along with the overall sensor device, it is appreciated that the system is now able to treat any event while the device is not being worn as a false event. Second, the system can use the proximity sensor for power management, powering down the headband when not worn, and powering the headband up automatically when the headband is placed on the head. In this way, battery life is extended.

It is appreciated that there are additional issues with using antennas within a headband or other type of worn device. In particular, it is appreciated that both the antenna used for connecting the headband to the network and the capacitive proximity sensor require relatively large antennas for optimal operation. Antennas extending from the mechanical package of the headband are prone to breakage. To protect these devices, one implementation may include embedding both the antenna (900 MHz or 2.4 GHz) and capacitive sensor in the flex circuit that connects the rigid portions of the headband. An example implementation is shown in FIG. 14.

The phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A sensor comprising:
a flexible housing comprising a plurality of 3-axis accelerometers, the flexible housing being adapted to be worn by a user and being in contact with a body part of the user; and
a module coupled to the accelerometers including a processor which is adapted to sense forces experienced by the body part.

2. The sensor device according to claim 1, wherein the sensor is constructed within a headband.

3. The sensor device according to claim 1, wherein the flexible housing comprises at least three 3-axis accelerometers, and wherein the housing comprises flexible couplings between the at least three 3-axis accelerometers.

4. The sensor device according to claim 3, further comprising flexible circuits that couple the module to two of the at least three 3-axis accelerometers.

5. The sensor device according to claim 1, wherein at least one of the 3-axis accelerometers is positioned at the back of the head of the user in alignment with the median nuchal line.

6. The sensor according to claim 1, wherein the module is adapted to determine rotational acceleration from multiple measurements of linear acceleration.

7. The sensor according to claim 6, wherein the module is adapted to receive at least three measurements of linear acceleration from non-colinear points within the sensor.

8. The sensor according to claim 6, further comprising a sensor adapted to measure rotational velocity.

9. The sensor according to claim 8, wherein the sensor is adapted to measure rotational velocity includes a MEMS gyro.

10. The sensor according to claim 9, wherein the module is adapted to power on the MEMS gyro responsive to a detection of an event.

11. The sensor according to claim 10, wherein the event includes a measurement of an acceleration above a predefined threshold.

12. The sensor according to claim 1, further comprising a proximity sensor adapted to detect whether the sensor is in contact with the body part of the user.

13. The sensor according to claim 12, wherein the module is adapted to power on the sensor responsive to detection by the proximity sensor indicates that sensor is being worn by the user.

14. The sensor according to claim 12, wherein the proximity sensor includes a capacitive sensor pad.

15. The sensor according to claim 1, wherein the housing includes a plurality of rigid circuits associated with each of the plurality of 3-axis accelerometers.

16. The sensor according to claim 15, wherein the plurality of rigid circuits are coupled by a plurality of flexible circuits.

17. The sensor according to claim 1, further comprising, within the flexible housing, at least one antenna adapted to communicate with one or more external systems.

18. The sensor according to claim 1, further comprising, within the flexible housing, a capacitive sensor pad adapted to detect proximity of the sensor to the body part of the user.

19. The sensor according to claim 1, wherein at least one of the plurality of 3-axis accelerometers is coupled to a rigid circuit associated with the module, the at least one accelerometer being coupled to the rigid circuit by a flexible circuit.
